(19) United States
(12) Patent Application Publication
(10) Pub. No.: US 2007/0276229 A1
(43) Pub. Date: Nov. 29, 2007

(54) Breast restraint

(75) Inventor: John R. Adler, Stanford, CA (US)

Correspondence Address: BLAKELY SOKOLOFF TAYLOR & ZAFMAN, 1279 OAKMEAD PARKWAY, SUNNYVALE, CA, 94085-4040, US

(73) Assignee: Accuray Incorporated

(21) Appl. No.: 11/440,143

(22) Filed: May 23, 2006

(51) Int. Cl. A61B 6/00; G21K 5/08

(52) U.S. Cl. 600/426; 378/68

(57) ABSTRACT

A method and apparatus to immobilize a breast of a patient and position a fiducial about the exterior of the breast. The apparatus includes a cup forming a cavity to accept the breast of the patient within the cup. The apparatus also includes a fiducial marker coupled to the cup. The fiducial marker has a spatial relationship with a target region of the breast.

### TECHNICAL FIELD

This invention relates to the field of radiation treatment and, in particular, to a breast restraint to reliably immobilize a breast of a patient and consistently position a fiducial about the exterior of the breast.

### BACKGROUND

Pathological anatomies such as tumors and lesions can be treated with an invasive procedure, like surgery, which has significant risks for the patient. A non-invasive method to treat a pathological anatomy or other target is external beam radiation therapy. A “target” as discussed herein may be an anatomical feature(s) of a patient such as a pathological anatomy (e.g., tumor, lesion, vascular malformation, nerve disorder, etc.) or normal anatomy and may include one or more non-anatomical reference structures. In one type of external beam radiation therapy, an external radiation source is used to direct a sequence of x-ray beams at a tumor site from multiple angles, with the patient positioned so the tumor is at the center of rotation (isocenter) of the beam. As the angle of the radiation source changes, every beam passes through the tumor site, but passes through a different area of healthy tissue on its way to the tumor. As a result, the cumulative radiation dose at the tumor is high and the average radiation dose to healthy tissue is low.

The term “radiotherapy” refers to a procedure in which radiation is applied to a target region for therapeutic, rather than necrotic, purposes. The amount of radiation utilized in a single radiotherapy session is typically about an order of magnitude smaller than the amount used in a radiosurgery session. Radiotherapy is typically characterized by a low dose per treatment (e.g., 100-200 centiGray (cGy)), short treatment times (e.g., 10 to 30 minutes per treatment) and hyperfractionation (e.g., 30 to 45 days of treatment). Radiosurgery treatment typically lasts 30 minutes to 2 hours and involves from 1 to 5 sessions. For convenience, the term “radiation treatment” is used herein to include radiosurgery and/or radiotherapy unless otherwise noted.

Conventional radiation treatment can be divided into at least two distinct phases: treatment planning and treatment delivery. A treatment planning system may be employed to develop a treatment plan to deliver a requisite dose to a target region, while minimizing exposure to healthy tissue and avoiding sensitive critical structures. A treatment delivery system may be employed to deliver the radiation treatment according to the treatment plan. Treatment plans specify quantities such as the directions and intensities of the applied radiation beams, and the durations of the beam exposure. A treatment plan may be generated from input parameters such as beam positions, beam orientations, beam shapes, beam intensities, and radiation dose distributions (which are typically deemed appropriate by the radiologist in order to achieve a particular clinical goal). Sophisticated treatment plans may be developed using advanced modeling techniques and optimization algorithms.

Two kinds of treatment planning procedures are conventionally known: forward planning and inverse planning. In forward treatment planning, a medical physicist determines the radiation dose of a chosen beam and then calculates how much radiation will be absorbed by the tumor, critical structures (i.e., vital organs), and other healthy tissue. There is no independent control of the dose levels to the tumor and other structures for a given number of beams, because the radiation absorption in a volume of tissue is determined by the properties of the tissue and the distance of each point in the volume to the origin of the beam and the beam axis. The treatment planning system then calculates the resulting dose distribution and the medical physicist may iteratively adjust the values of the treatment parameters during treatment planning until an adequate dose distribution is achieved.

In contrast, the medical physicist may employ inverse planning to specify the minimum dose to the tumor and the maximum dose to other healthy tissues independently, and the treatment planning system then selects the direction, distance, and total number and intensity of the beams in order to achieve the specified dose conditions. Given a desired dose distribution specified and input by the user (e.g., the minimum and maximum doses), the inverse planning module selects and optimizes dose weights and/or beam directions, i.e. select an optimum set of beams that results in such a distribution. Inverse planning may have the advantage of being able to produce better plans, when used by less sophisticated users.

Implementing the treatment plan at the time of treatment delivery may be difficult because the treatment delivery conditions may be different from the treatment planning conditions. Thus, modeling established during the treatment planning stage may not be useful, unless fiducial landmarks used during the treatment planning to model the position of the tumor are repositioned in the same relative location during treatment delivery. Although internal fiducials may be implanted into or around the tumor, such invasive procedures may be minimized or avoided by using external fiducials. However, the placement of external fiducials may require great care to ensure that the fiducials are worn or located in the same position as during pre-treatment imaging.

The ideal radiation procedure delivers as much radiation as possible to the pathologic tissue and as little radiation (as physically possible) to the surrounding normal tissue. Such a procedure benefits from a very accurate targeting of a large number of radiation beams. Markers that have a fixed relationship with the target lesion and which can be visualized at the time of irradiation are a well known technique used to precisely aim a radiation beam. However, the use of markers for targeting soft tissue lesions such as breast tumors is problematic. By nature, the breast is a largely amorphous structure (more so in older women) that is difficult to exactly reposition. It is therefore difficult to reliably get the breast in the same shape and position at the time of imaging prior to radiation therapy and at the time of actual breast irradiation.

While a vest may hold the breast tissue in a relatively fixed position, compressing the breast tissue against the chest wall complicates radiation delivery because the tumor may be relatively close to other organs within the patient's chest. Rather than compressing the breast tissue against the chest wall, it would be safer and easier to deliver radiation to the target region within the breast while the breast tissue is protracted, or pulled away, from the chest wall. However, maintaining the breast tissue in such a position may be difficult because of the lack of structure within the breast. Furthermore, even if the breast is maintained in a protracted position, there is no conventional technology to facilitate reproducible fiducial placement with reference to a tumor within the breast.

### BRIEF DESCRIPTION OF THE DRAWINGS

The present invention is illustrated by way of example, and not by way of limitation, in the figures of the accompanying drawings.

FIG. 1 illustrates one embodiment of a breast restraint.

FIG. 2 illustrates one embodiment of an immobilization apparatus.

FIG. 3 illustrates one embodiment of an application position of a patient to facilitate initial positioning of the immobilization apparatus.

FIG. 4A illustrates one embodiment of a treatment position in which a patient wears the immobilization apparatus during radiation treatment.

FIG. 4B illustrates another treatment position in which a patient wears another embodiment of immobilization apparatus with vacuum suction to pull the breast away from the chest wall during radiation treatment.

FIG. 5 illustrates one embodiment of a pre-treatment method for using the immobilization apparatus.

FIG. 6 illustrates one embodiment of a treatment method for using the immobilization apparatus.

FIG. 7 illustrates one embodiment of a treatment system that may be used to perform radiation treatment in which embodiments of the present invention may be implemented.

FIG. 8 is a schematic block diagram illustrating one embodiment of a treatment delivery system.

FIG. 9 illustrates a three-dimensional perspective view of a radiation treatment process.

### DETAILED DESCRIPTION

The following description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Thus, the specific details set forth are merely exemplary. Particular implementations may vary from these exemplary details and still be contemplated to be within the spirit and scope of the present invention.

One embodiment of an immobilization is described. The immobilization apparatus includes a cup having a cavity configured to accept a breast of a patient within the cup. The cup is shaped to hold the breast in a protracted position away from a chest wall of the patient. A fiducial marker, which has a spatial relationship with a target region of the breast, is coupled to the cup. Embodiments of the immobilization apparatus also may include an alignment marker on the cup to facilitate alignment of the cup on the breast in relation to an alignment landmark on the breast. Exemplary alignment landmarks include a tattoo on the breast, a nib such as a multi-modality nib attached to the breast, and the nipple of the breast. Another embodiment of the immobilization apparatus also includes a strap coupled to the cup to hold the cup on the breast. The strap may include a compression portion to compress an adjacent breast of the patient against the chest wall of the patient. Another embodiment of the immobilization apparatus also includes a vacuum coupled to the cup to pull the breast away from a chest wall of the patient. Additional embodiments of the immobilization apparatus are also described.

A method for using an immobilization apparatus is also described. An embodiment of the method includes positioning a cup, including a fiducial marker, on a breast of a patient and determining a relationship between the fiducial marker and a target region of the breast. The relationship of the fiducial marker and the target region may be determined during a treatment planning session, and then the immobilization apparatus may be removed. Later, the immobilization apparatus may be realigned on the breast so that the fiducial marker and the target region resume the same relationship as during the treatment planning session. In order to consistently realign the immobilization apparatus on the breast, an embodiment of the method includes marking a tattoo on the breast through a marking aperture in the cup before removing the cup from the breast. Another embodiment of the method includes gluing a nib on the breast to align with a congruent marking aperture in the cup. Another embodiment of the method includes aligning a marking aperture of the cup with the nipple of the breast. Consistently realigning the cup on the breast facilitates positioning a radiation source relative to the target region based on a location of the fiducial marker and reliably irradiating the target region with a radiation beam from the radiation source. Additional embodiments of the immobilization method are also described.

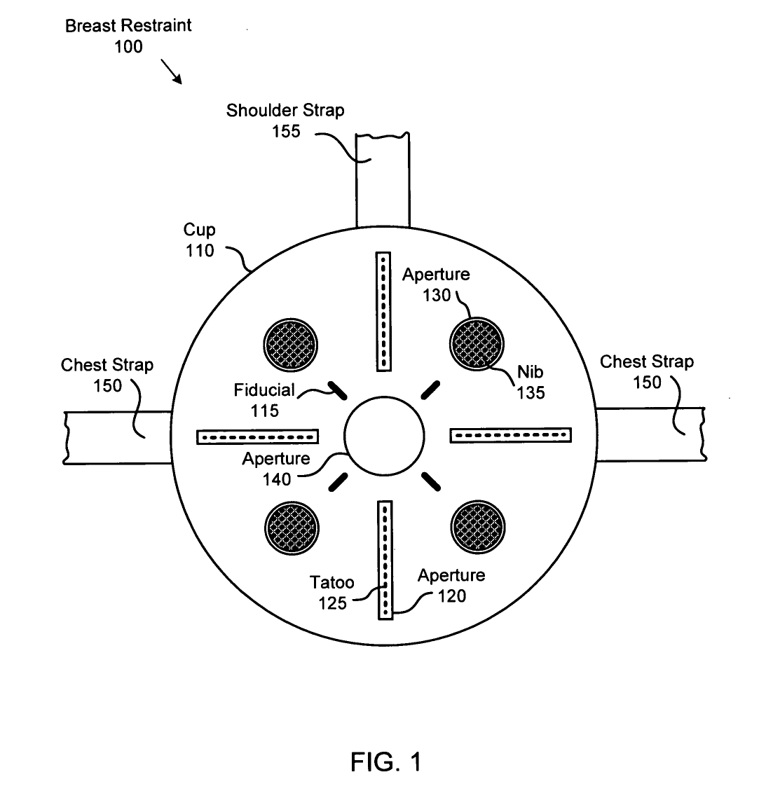

FIG. 1 illustrates one embodiment of a breast restraint 100. In particular, a front view of the breast restraint 100 is illustrated. In general, the breast restraint 100 may function similar to a brassiere to support one or both of a female's breasts in a defined position. Although this description describes the functionality of the breast restraint 100 in terms of radiation of a tumor within a female's breast, other anatomical restraints may be implemented to perform similar functions in regard to other anatomical appendages which lack substantial structure. Moreover, certain embodiments may be implemented to perform similar functions in regard to a male's breasts or other body parts. Therefore, the term patient is used herein to inclusively refer to either female or male patients, unless otherwise indicated.

The depicted breast restraint 100 includes a cup 110. In some aspects, the cup 110 is similar to the cup of a brassiere in that the cup 110 forms a cavity to hold a breast. Different sizes of cups 110 may be used for different sizes of breasts. In some embodiments, the cup 110 may be formed of a rigid or semi-rigid material such as a translucent plastic so that the cup 110 substantially maintains its shape when it is worn by a patient. Alternatively, the cup 110 may have another structure such as mesh, cloth, or wire frame to provide rigidity to the cup 110. In another embodiment, the cup 110 is formed of a radiolucent material so that a radiation beam may pass through the cup 110 to irradiate a target region within the breast. Additionally, the cup 110 may have other materials applied to the interior or exterior of the cup 110. For example, a bolus material may be applied to the interior of the cup 110 to alter the effective depth of the radiation treatment.

In one embodiment, the breast restraint 100 includes one or more fiducials 115. The fiducials 115 are radiopaque so that they are visible using a radiation imaging system. For example, the fiducials 115 may be gold seeds. Alternatively, the fiducials 115 may be visible using an ultrasound or other imaging system. The fiducials 115 may be attached to or integrated into the cup 110 in any manner so that they maintain a relatively fixed position on the cup 110. Although the fiducials 115 are shown in a particular pattern, other embodiments may implement the breast restraint 100 with fewer or more fiducials 115 in a similar or different arrangement.

In one embodiment, a model may be established to describe a spatial relationship or physical correlation between one or more fiducials 115 and a target region within the breast. The model or correlation may be determined prior to delivery of radiation treatment. This correlation may be used during radiation treatment to position a radiation source and direct a radiation beam at the target region based on the position of the fiducials 115. For example, an imaging system may determine the position of the fiducials 115 and control a radiation source so that a radiation beam irradiates a tumor within the breast. In this way, the location of the target region may be determined based on the locations of the external fiducials 115 without the use of internal fiducials.

The breast restraint 100 also may include one or more alignment elements or landmarks. In general, an alignment element may be used to facilitate consistent alignment of the cup 110 on the breast at the time of pretreatment imaging or mapping and for multiple treatment sessions. After initially developing a model to describe the relationship between the fiducials 115 and the target region within the breast, the breast restraint 100 is removed until the first radiation treatment session begins. In order to benefit from the accuracy of the model, the breast restraint 100 may be aligned in the same position it was in during the initial pre-treatment modeling. This position is referred to herein as the alignment position. Given the compliance of the breast, consistent realignment of the cup 110 on the breast may be difficult without the use of alignment elements.

One example of an alignment element is a nib aperture 130. The nib aperture 120 may be used to engage a nib 135 applied to the skin of the breast. As used herein, a nib 135 may be any type of small object, not limited to a particular shape or configuration, which may be applied to the breast. In one embodiment, the nib 135 may be glued to the skin, although the nib 135 may be attached in other ways. One exemplary nib 135 is a fiducial nib which functions as a fiducial as well as an alignment nib 135. In one embodiment, the nibs 135 may be glued onto the skin of the breast accessible through the corresponding nib apertures 130 during a pre-treatment session. Although a particular number and configuration of nib apertures 130 and nibs 135 are shown, other embodiments may facilitate fewer or more nib apertures 130, alternative arrangements and shapes, and so forth.

Another example of an alignment element is a tattoo aperture 120. The tattoo aperture 120 may be used to access the skin of the breast through the cup 110 to apply a tattoo 125 or other marking on the skin. Through the use of one or more tattoos 125 applied at a corresponding number of tattoo apertures 120, the cup 110 may be realigned on the breast (e.g., during a subsequent treatment session) by lining up the tattoos 125 with the corresponding tattoo apertures 120. In one embodiment, permanent or semi-permanent tattoos 125 which persist during at least the time between treatment sessions may be used. Although a particular number and configuration of tattoo apertures 120 and tattoos 125 are shown, other embodiments may facilitate fewer or more tattoos 125, alternative arrangements and shapes, and so forth. In one embodiment, the position of the breast within the cup is adjusted to maximize tattoo alignment.

In one embodiment, the cup 110 also may include a nipple aperture 140. The nipple aperture 140 allows the nipple of the breast to protrude through the cup 110. In this way, the nipple also may be used as a landmark in aligning the cup 110 on the breast. In other embodiments, other types of natural or artificial alignment landmarks may be used, including the areola of the nipple, birthmarks and moles on the breast, ridges or markings in the cup 110, and so forth. Furthermore, although the depicted breast restraint 100 includes various types of marker elements, other embodiments of the breast restraint 100 may include fewer or more marker elements, as well as other combinations of marker elements.

In one embodiment, the cup 110 may be attached to one or more straps to retain the cup 110 on the patient's breast. The straps may be similar to straps on a brassiere. For example, the breast restraint 100 may include a chest strap 150 to go around the patient's chest. In another embodiment, the breast restraint 100 may include a shoulder strap to go over the patient's shoulder. Alternatively, the breast restraint 100 may be maintained on the breast (or the breast maintained in the cup 110) through the use of an adhesive, a vacuum, or another retention mechanism.

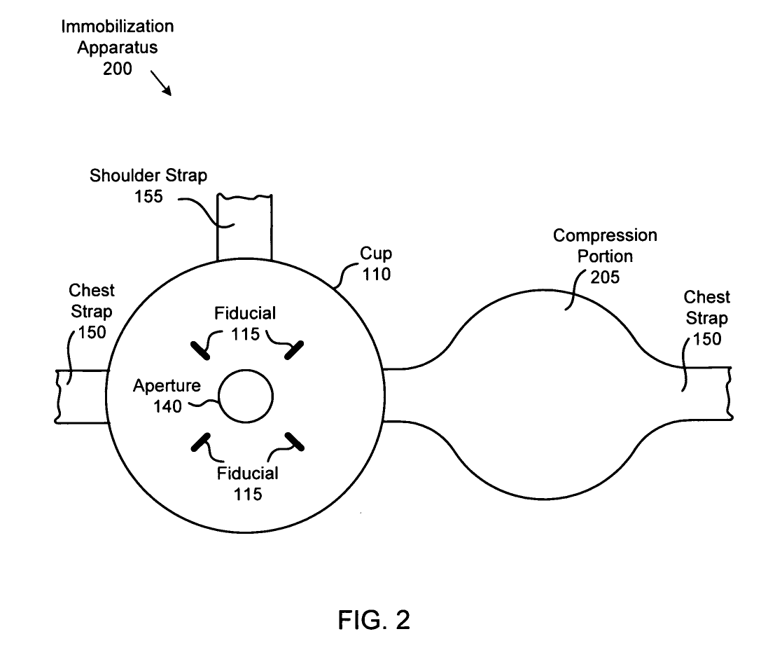

FIG. 2 illustrates one embodiment of an immobilization apparatus 200. In one embodiment, the immobilization apparatus 200 includes a breast restraint which is similar to the breast restraint 100 of FIG. 1. The immobilization apparatus 200 includes a cup 110, several fiducials 115, and a nipple aperture 140. Other features of the cup 110 are omitted for clarity, although other embodiments of the immobilization apparatus 200 may include other breast restraints with different features. The immobilization apparatus 200 also includes a chest strap 150 and a shoulder strap 155, as described above with reference to FIG. 1.

In one embodiment, the chest strap 150 may include a compression portion 205. The compression portion 205 is approximately located over an adjacent breast (e.g., the breast that is not being irradiated). In one embodiment, the compression portion 205 of the chest strap 150 compresses, or flattens, the adjacent breast. In this manner, the adjacent breast may be pressed against the patient's chest in order to maximize the number of positions from which the radiation source may irradiate the target region of the restrained breast. Alternatively, the compression portion 205 may be configured to move the adjacent breast toward the patient's side, toward the patient's stomach, or in another direction to decrease radiation administered to the healthy breast. By maintaining the restrained breast in a protracted position (e.g., elevated when the patient is lying on her back) and moving the adjacent breast away from the restrained breast, a radiation source such as a stereotactic linear accelerator (LINAC) may have relatively more nodes from which to deliver radiation treatment to the target region of the restrained breast.

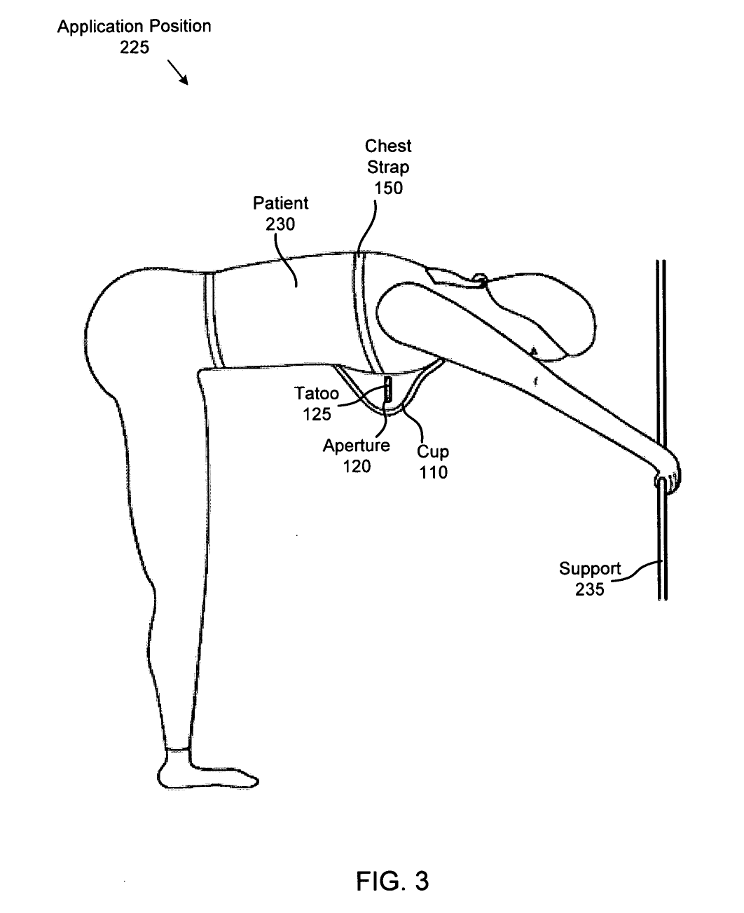

FIG. 3 illustrates one embodiment of an application position 225 of a patient 230 to facilitate initial positioning of the immobilization apparatus 200 on the breast. In one embodiment, the immobilization apparatus 200 is put on the patient 230 while the patient 230 is in a prone position (i.e., face down). A support 235 may be provided to stabilize the patient 230 while the immobilization device 200 is positioned and secured. In this manner, the breast is allowed to freely hang down from the patient's chest and assumes a reproducible shape. This hanging position may be referred to as a protracted position because the breast tissue is substantially protracted away from the chest wall. The cup 110 may be formed in a manner and of a material which, when worn by the patient, maintains the protracted position of the breast. Maintaining the breast in the protracted position during radiation treatment may increase the distance between the radiation beam and the patient's body, thereby making the radiation treatment relatively safer for the patient. Additionally, maintaining the breast in the protracted position during radiation treatment may allow the radiation source to be positioned at more delivery nodes than might be available if the breast were in a position near the chest wall.

At pre-treatment and pre-imaging sessions, while the immobilization apparatus 200 is worn by the patient, a physician or other practitioner may apply the nibs 135, tattoos 125, or mark other landmarks so that the immobilization apparatus 200 may be removed and worn again later in the aligned position. At subsequent pre-treatment or treatment sessions, the immobilization apparatus 200 may be put on in a similar manner as during the initial pre-treatment session so that the alignment position may be replicated.

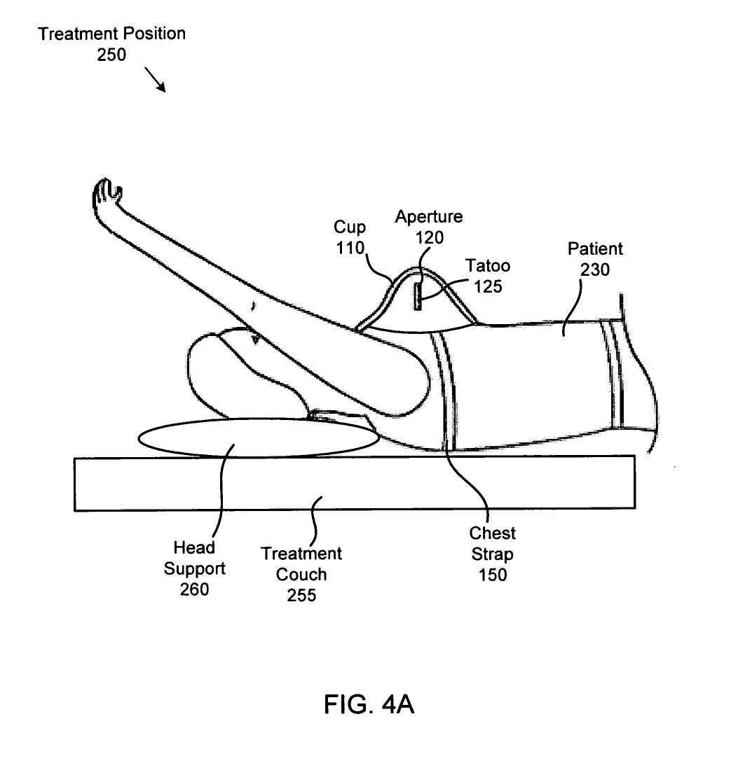

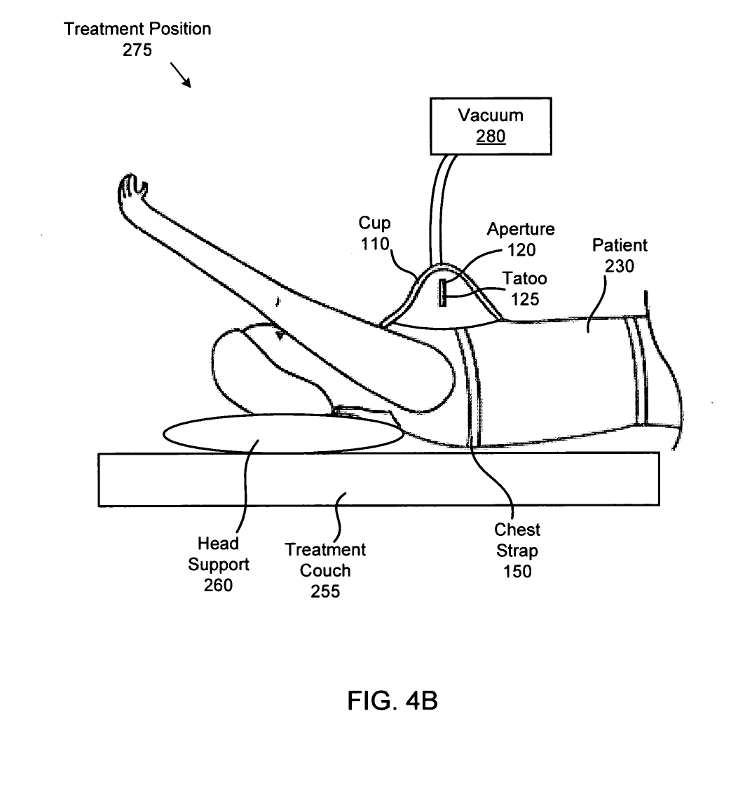

FIG. 4A illustrates one embodiment of a treatment position 250 of a patient 230 to wear the immobilization apparatus 200 during radiation treatment. As explained above, the immobilization apparatus 200 may maintain the restrained breast in a protracted position (also referred to as an elevated position when the patient is lying on her back). Additionally, the immobilization apparatus 200 may maintain the adjacent breast in a position away from the potential radiation beam paths.

Although the patient 230 may be positioned during radiation in several ways, one exemplary position is in the supine position (i.e., face up). For example, the patient 230 may lie on her back on a treatment couch 255. A head support 260 may be provided to support the patient's head. Additionally, an arm support may be provided for one or both of the patient's arms so that the arms are away from the restrained breast and general area of radiation treatment. In other embodiments, other types of patient supports may be provided. Alternatively, the patient 230 may be treated in another position. For example the patient 230 may be positioned on her side, on her stomach (e.g., where the breast is allowed to protrude through a cutout or hole in the treatment couch 255), or in a seated or standing position. An advantage of one embodiment is that the restrained breast may be maintained in the protracted position regardless of the treatment position of the patient 230.

FIG. 4B illustrates another treatment position 275 of a patient 230 to wear another embodiment of immobilization apparatus 200 with vacuum suction to pull the breast away from the chest wall during radiation treatment. Although the immobilization apparatus 200 of FIG. 4B is similar in many aspects to the immobilization apparatus 200 of FIG. 4A, the immobilization apparatus 200 of FIG. 4B includes a vacuum 280 to apply vacuum suction to the cup 110 in order to further influence the breast away from the chest wall. While the cup 110 alone may provide sufficient rigidity to maintain the position of the breast, the vacuum 280 may facilitate further immobility of the breast within the cup 110. In another embodiment, the vacuum 280 also facilitates increased protraction of the breast away from the chest wall.

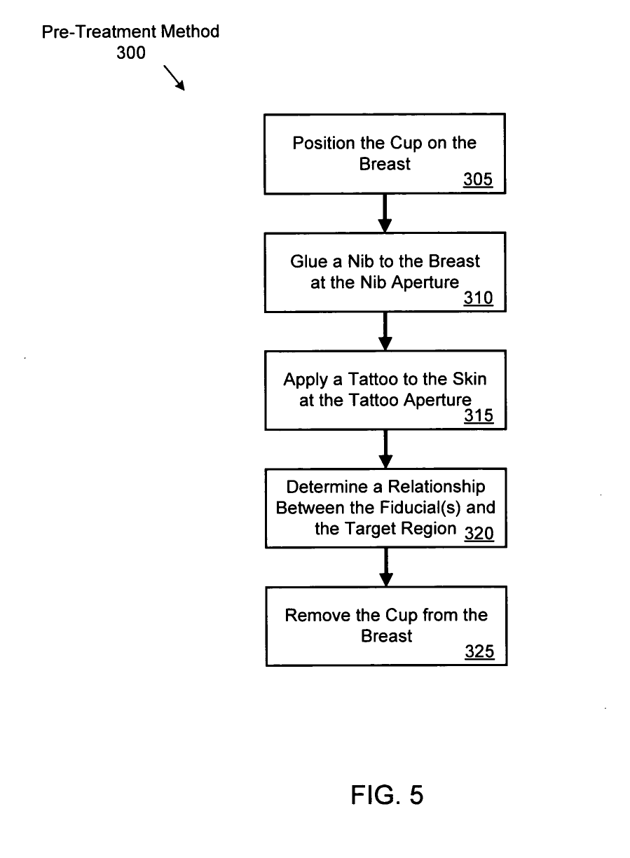

FIG. 5 illustrates one embodiment of a pre-treatment method 300 for using the immobilization apparatus 200. To begin, a physician or other medical practitioner may position 305 the cup 110 of the immobilization apparatus 200 on the patient 230. Alternatively, the immobilization apparatus 200 may be configured to allow the patient 230 to put on the immobilization apparatus 200 by herself. The chest strap 150 and shoulder strap 155 may be secured to the patient 230 to maintain the cup 110 on the breast. The physician or medical practitioner then may glue 310 or otherwise adhere a nib 135 to the breast at a nib aperture 130. As described above, the nib 135 may function as an alignment landmark for reproducibly aligning the cup 110 on the breast and/or as a fiducial for determining a spatial relationship between the nib 135 and the target region to be irradiated. Subsequently, the physician or medical practitioner may apply 315 a tattoo 125 to the restrained breast through a tattoo aperture 120. The tattoo 125 may be temporary, semi-permanent (e.g., use of a “permanent” marker), or permanent (e.g., micro-pigment implantation). In another embodiment, the physician or medical practitioner also may implement other markings or landmarks in addition to the tattoo 125 and nib 135.

After the cup 110 and breast are aligned and marked in a manner to facilitate subsequent realignment of the cup 110 on the breast, the physician or medical practitioner may determine 320 a spatial relationship between the fiducials 115 attached to the cup 110 (and potentially the nibs 135) and the target region of the breast. For example, the medical practitioner may use imaging such as CT scanning to determine 320 the spatial relationship between the fiducials 115 and the target region. In one embodiment, this relationship correlates the locations of the target region to the locations of the fiducials 115 at specified points in time so that the location of the target region may be determined during treatment delivery based on the known locations of the fiducials 115. This correlation may be expressed as a model and may account for dynamic movement of the target region and/or fiducials 115. Such dynamic movement may result from the respiratory cycle of the patient 230, the cardio cycle of the patient 230, positioning adjustments of the patient 230, and so forth. After the relationship between the fiducials 115 and the target region is established, the immobilization apparatus 200 and cup 110 may be removed from the patient 230. The illustrated pre-treatment method 300 then ends.

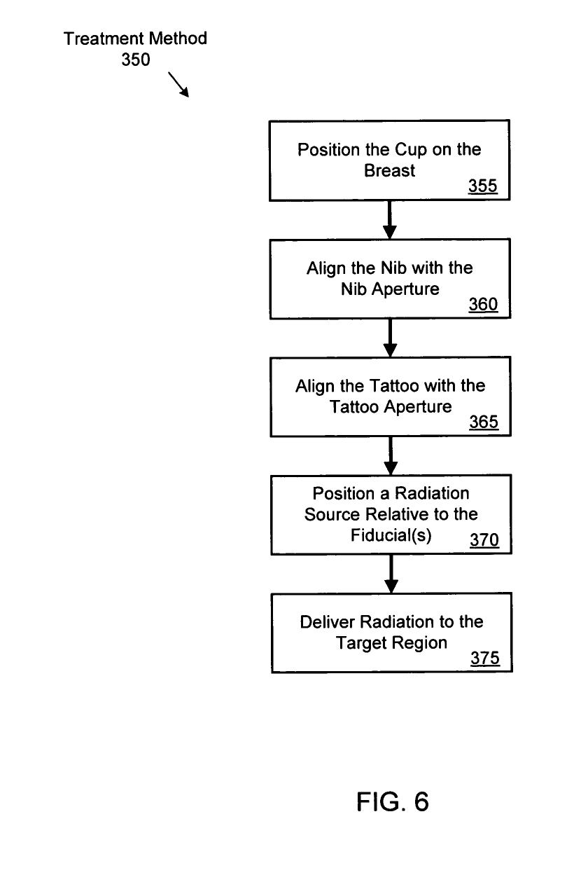

FIG. 6 illustrates one embodiment of a treatment method 350 for using the immobilization apparatus 200. To begin, the physician or medical practitioner positions 355 the cup 110 and immobilization apparatus 200 on the patient 230. In order to benefit from the correlation established during the pre-treatment session or another earlier session, the physician or medical practitioner positions 355 the cup 110 in the aligned position according to the marks and landmarks implemented in the pre-treatment session. For example, the physician or medical practitioner may align 360 the nib 135 with the corresponding nib aperture 130. Similarly, the physician or medical practitioner may align 365 the tattoo 125 with the corresponding tattoo aperture 120. In this way, the cup 110 may be reproducibly positioned on the breast in a similar orientation and relationship to the target region of the breast as during the pre-treatment session.

After putting the immobilization apparatus 200 on the patient 230, the physician or medical practitioner may proceed to position 370 a radiation source relative to the known locations of the fiducials 115. In one embodiment, diagnostic imaging may be used to determine the locations of the fiducials 115 during treatment delivery, although other imaging techniques may be used. With the radiation source positioned to irradiate the target region based on the locations of the fiducials 115, the physician or medical practitioner then delivers 375 radiation to the target region according to a treatment plan. The illustrated treatment method 350 then ends.

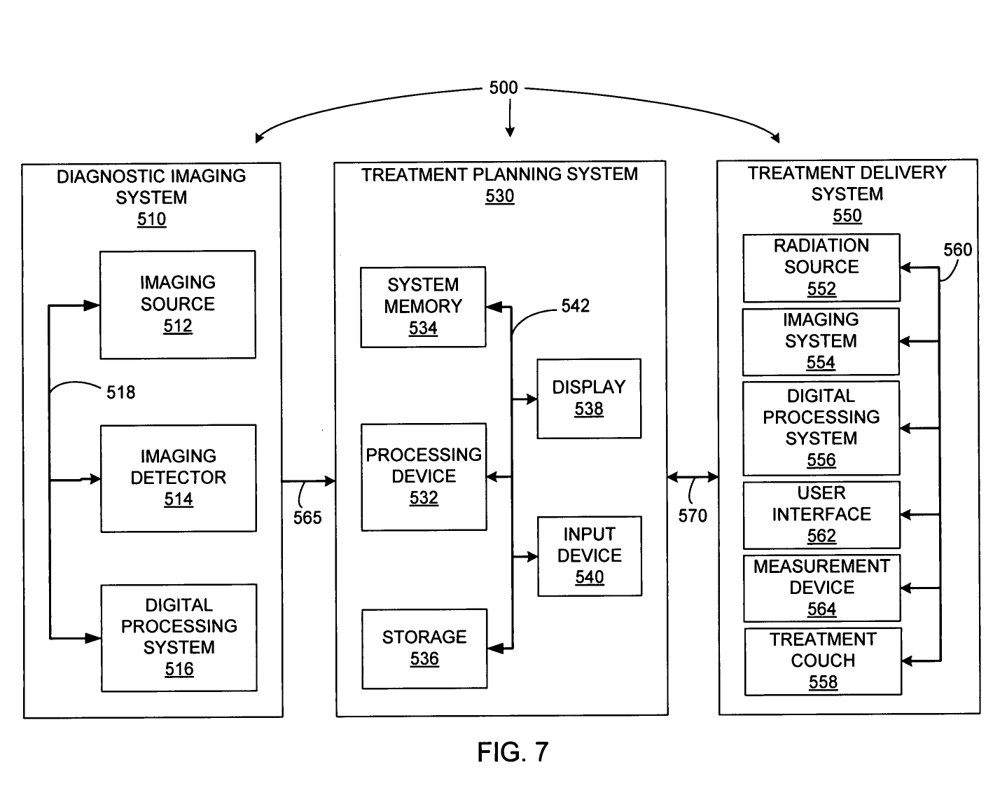

FIG. 7 illustrates one embodiment of a treatment system 500 that may be used to perform radiation treatment in which embodiments of the present invention may be implemented. The depicted treatment system 500 includes a diagnostic imaging system 510, a treatment planning system 530, and a treatment delivery system 550. In other embodiments, the treatment system 500 may include fewer or more component systems.

The diagnostic imaging system 510 is representative of any system capable of producing medical diagnostic images of a volume of interest (VOI) in a patient, which images may be used for subsequent medical diagnosis, treatment planning, and/or treatment delivery. For example, the diagnostic imaging system 510 may be a computed tomography (CT) system, a single photon emission computed tomography (SPECT) system, a magnetic resonance imaging (MRI) system, a positron emission tomography (PET) system, a near infrared fluorescence imaging system, an ultrasound system, or another similar imaging system. For ease of discussion, any specific references herein to a particular imaging system such as a CT x-ray imaging system (or another particular system) is representative of the diagnostic imaging system 510, generally, and does not preclude other imaging modalities, unless noted otherwise.

The illustrated diagnostic imaging system 510 includes an imaging source 512, an imaging detector 514, and a digital processing system 516. The imaging source 512, imaging detector 514, and digital processing system 516 are coupled to one another via a communication channel 518 such as a bus. In one embodiment, the imaging source 512 generates an imaging beam (e.g., x-rays, ultrasonic waves, radio frequency waves, etc.) and the imaging detector 514 detects and receives the imaging beam. Alternatively, the imaging detector 514 may detect and receive a secondary imaging beam or an emission stimulated by the imaging beam from the imaging source (e.g., in an MRI or PET scan). In one embodiment, the diagnostic imaging system 510 may include two or more diagnostic imaging sources 512 and two or more corresponding imaging detectors 514. For example, two x-ray sources 512 may be disposed around a patient to be imaged, fixed at an angular separation from each other (e.g., 90 degrees, 45 degrees, etc.) and aimed through the patient toward corresponding imaging detectors 514, which may be diametrically opposed to the imaging sources 514. A single large imaging detector 514, or multiple imaging detectors 514, also may be illuminated by each x-ray imaging source 514. Alternatively, other numbers and configurations of imaging sources 512 and imaging detectors 514 may be used.

The imaging source 512 and the imaging detector 514 are coupled to the digital processing system 516 to control the imaging operations and process image data within the diagnostic imaging system 510. In one embodiment, the digital processing system 516 may communicate with the imaging source 512 and the imaging detector 514. Embodiments of the digital processing system 516 may include one or more general-purpose processors (e.g., a microprocessor), special purpose processors such as a digital signal processor (DSP), or other type of devices such as a controller or field programmable gate array (FPGA). The digital processing system 516 also may include other components (not shown) such as memory, storage devices, network adapters, and the like. In one embodiment, the digital processing system 516 generates digital diagnostic images in a standard format such as the Digital Imaging and Communications in Medicine (DICOM) format. In other embodiments, the digital processing system 516 may generate other standard or non-standard digital image formats.

Additionally, the digital processing system 516 may transmit diagnostic image files such as DICOM files to the treatment planning system 530 over a data link 560. In one embodiment, the data link 560 may be a direct link, a local area network (LAN) link, a wide area network (WAN) link such as the Internet, or another type of data link. Furthermore, the information transferred between the diagnostic imaging system 510 and the treatment planning system 530 may be either pulled or pushed across the data link 560, such as in a remote diagnosis or treatment planning configuration. For example, a user may utilize embodiments of the present invention to remotely diagnose or plan treatments despite the existence of a physical separation between the system user and the patient.

The illustrated treatment planning system 530 includes a processing device 532, a system memory device 534, an electronic data storage device 536, a display device 538, and an input device 540. The processing device 532, system memory 534, storage 536, display 538, and input device 540 may be coupled together by one or more communication channel 542 such as a bus.

The processing device 532 receives and processes image data. The processing device 532 also processes instructions and operations within the treatment planning system 530. In certain embodiments, the processing device 532 may include one or more general-purpose processors (e.g., a microprocessor), special purpose processors such as a digital signal processor (DSP), or other types of devices such as a controller or field programmable gate array (FPGA).

In particular, the processing device 532 may be configured to execute instructions for performing treatment operations discussed herein. For example, the processing device 532 may identify a non-linear path of movement of a target within a patient and develop a non-linear model of the non-linear path of movement. In another embodiment, the processing device 532 may develop the non-linear model based on a plurality of position points and a plurality of direction indicators. In another embodiment, the processing device 532 may generate a plurality of correlation models and select one of the plurality of models to derive a position of the target. Furthermore, the processing device 532 may facilitate other diagnosis, planning, and treatment operations related to the operations described herein.

In one embodiment, the system memory 534 may include random access memory (RAM) or other dynamic storage devices. As described above, the system memory 534 may be coupled to the processing device 532 by the communication channel 542. In one embodiment, the system memory 534 stores information and instructions to be executed by the processing device 532. The system memory 534 also may be used for storing temporary variables or other intermediate information during execution of instructions by the processing device 532. In another embodiment, the system memory 534 also may include a read only memory (ROM) or other static storage device for storing static information and instructions for the processing device 532.

In one embodiment, the storage 536 is representative of one or more mass storage devices (e.g., a magnetic disk drive, tape drive, optical disk drive, etc.) to store information and instructions. The storage 536 and/or the system memory 534 also may be referred to as machine readable media. In a specific embodiment, the storage 536 may store instructions to perform the modeling operations discussed herein. For example, the storage 536 may store instructions to acquire and store data points, acquire and store images, identify non-linear paths, develop linear and/or non-linear correlation models, and so forth. In another embodiment, the storage 536 may include one or more databases.

In one embodiment, the display 538 may be a cathode ray tube (CRT) display, a liquid crystal display (LCD), or another type of display device. The display 538 displays information (e.g., a two-dimensional or three-dimensional representation of the VOI) to a user. The input device 540 may include one or more user interface devices such as a keyboard, mouse, trackball, or similar device. The input device(s) 540 may also be used to communicate directional information, to select commands for the processing device 532, to control cursor movements on the display 538, and so forth.

Although one embodiment of the treatment planning system 530 is described herein, the described treatment planning system 530 is only representative of an exemplary treatment planning system 530. Other embodiments of the treatment planning system 530 may have many different configurations and architectures and may include fewer or more components. For example, other embodiments may include multiple buses, such as a peripheral bus or a dedicated cache bus. Furthermore, the treatment planning system 530 also may include Medical Image Review and Import Tool (MIRIT) to support DICOM import so that images can be fused and targets delineated on different systems and then imported into the treatment planning system 530 for planning and dose calculations. In another embodiment, the treatment planning system 530 also may include expanded image fusion capabilities that allow a user to plan treatments and view dose distributions on any one of various imaging modalities such as MRI, CT, PET, and so forth. Furthermore, the treatment planning system 530 may include one or more features of convention treatment planning systems.

In one embodiment, the treatment planning system 530 may share a database on the storage 536 with the treatment delivery system 550 so that the treatment delivery system 550 may access the database prior to or during treatment delivery. The treatment planning system 530 may be linked to treatment delivery system 550 via a data link 570, which may be a direct link, a LAN link, or a WAN link, as discussed above with respect to data link 560. Where LAN, WAN, or other distributed connections are implemented, any of components of the treatment system 500 may be in decentralized locations so that the individual systems 510, 530 and 550 may be physically remote from one other. Alternatively, some or all of the functional features of the diagnostic imaging system 510, the treatment planning system 530, or the treatment delivery system 550 may be integrated with each other within the treatment system 500.

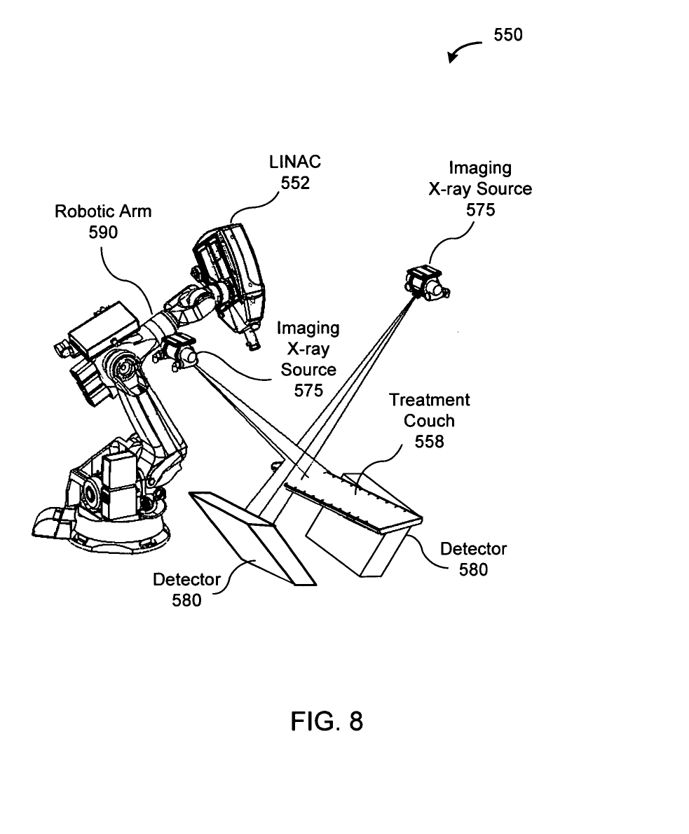

The illustrated treatment delivery system 550 includes a radiation source 552, an imaging system 554, a digital processing system 556, and a treatment couch 558. The radiation source 552, imaging system 554, digital processing system 556, and treatment couch 558 may be coupled to one another via one or more communication channels 560. One example of a treatment delivery system 550 is shown and described in more detail with reference to FIG. 8.

In one embodiment, the radiation source 552 is a therapeutic or surgical radiation source 552 to administer a prescribed radiation dose to a target volume in conformance with a treatment plan. For example, the target volume may be an internal organ, a tumor, a region. As described above, reference herein to the target, target volume, target region, target area, or internal target refers to any whole or partial organ, tumor, region, or other delineated volume that is the subject of a treatment plan.

In one embodiment, the imaging system 554 of the treatment delivery system 550 captures intra-treatment images of a patient volume, including the target volume, for registration or correlation with the diagnostic images described above in order to position the patient with respect to the radiation source. Similar to the diagnostic imaging system 510, the imaging system 554 of the treatment delivery system 550 may include one or more sources and one or more detectors.

The treatment delivery system 550 also may include a digital processing system 556 to control the radiation source 552, the imaging system 554, and a treatment couch 558, which is representative of any patient support device. The digital processing system 556 may include one or more general-purpose processors (e.g., a microprocessor), special purpose processors such as a digital signal processor (DSP), or other devices such as a controller or field programmable gate array (FPGA). Additionally, the digital processing system 556 may include other components (not shown) such as memory, storage devices, network adapters, and the like.

The illustrated treatment delivery system 550 also includes a user interface 562 and a measurement device 564. In one embodiment, the user interface 562 allows a user to interface with the treatment delivery system 550. In particular, the user interface 562 may include input and output devices such as a keyboard, a display screen, and so forth. The measurement device 564 may be one or more devices that measure external factors such as the external factors described above, which may influence the radiation that is actually delivered to the target region. Some exemplary measurement devices include a thermometer to measure ambient temperature, a hygrometer to measure humidity, a barometer to measure air pressure, or any other type of measurement device to measure an external factor.

FIG. 8 is a schematic block diagram illustrating one embodiment of a treatment delivery system 550. The depicted treatment delivery system 550 includes a radiation source 552, in the form of a linear accelerator (LINAC), and a treatment couch 558, as described above. The treatment delivery system 550 also includes multiple imaging x-ray sources 575 and detectors 580. The two x-ray sources 575 may be nominally aligned to project imaging x-ray beams through a patient from at least two different angular positions (e.g., separated by 90 degrees, 45 degrees, etc.) and aimed through the patient on the treatment couch 558 toward the corresponding detectors 580. In another embodiment, a single large imager may be used to be illuminated by each x-ray imaging source 575. Alternatively, other quantities and configurations of imaging sources 575 and detectors 580 may be used. In one embodiment, the treatment delivery system 550 may be an image-guided, robotic-based radiation treatment system (e.g., for performing radiosurgery) such as the CyberKnife® radiation treatment system developed by Accuray Incorporated of California.

In the illustrated embodiment, the LINAC 552 is mounted on a robotic arm 590. The robotic arm 590 may have multiple (e.g., 5 or more) degrees of freedom in order to properly position the LINAC 552 to irradiate a target such as a pathological anatomy with a beam delivered from many angles in an operating volume around the patient. The treatment implemented with the treatment delivery system 550 may involve beam paths with a single isocenter (point of convergence), multiple isocenters, or without any specific isocenters (i.e., the beams need only intersect with the pathological target volume and do not necessarily converge on a single point, or isocenter, within the target). Furthermore, the treatment may be delivered in either a single session (mono-fraction) or in a small number of sessions (hypo-fractionation) as determined during treatment planning. In one embodiment, the treatment delivery system 550 delivers radiation beams according to the treatment plan without fixing the patient to a rigid, external frame to register the intra-operative position of the target volume with the position of the target volume during the pre-operative treatment planning phase.

As described above, the digital processing system 556 may implement algorithms to register images obtained from the imaging system 554 with pre-operative treatment planning images obtained from the diagnostic imaging system 510 in order to align the patient on the treatment couch 558 within the treatment delivery system 550. Additionally, these images may be used to precisely position the radiation source 552 with respect to the target volume or target.

In one embodiment, the treatment couch 558 may be coupled to second robotic arm (not shown) having multiple degrees of freedom. For example, the second arm may have five rotational degrees of freedom and one substantially vertical, linear degree of freedom. Alternatively, the second arm may have six rotational degrees of freedom and one substantially vertical, linear degree of freedom. In another embodiment, the second arm may have at least four rotational degrees of freedom. Additionally, the second arm may be vertically mounted to a column or wall, or horizontally mounted to pedestal, floor, or ceiling. Alternatively, the treatment couch 558 may be a component of another mechanism, such as the AXUM® treatment couch developed by Accuray Incorporated of California. In another embodiment, the treatment couch 558 may be another type of treatment table, including a conventional treatment table.

Although one exemplary treatment delivery system 550 is described above, the treatment delivery system 550 may be another type of treatment delivery system. For example, the treatment delivery system 550 may be a gantry based (isocentric) intensity modulated radiotherapy (IMRT) system, in which a radiation source 552 (e.g., a LINAC) is mounted on the gantry in such a way that it rotates in a plane corresponding to an axial slice of the patient. Radiation may be delivered from several positions on the circular plane of rotation. In another embodiment, the treatment delivery system 550 may be a stereotactic frame system such as the GammaKnife®, available from Elekta of Sweden.

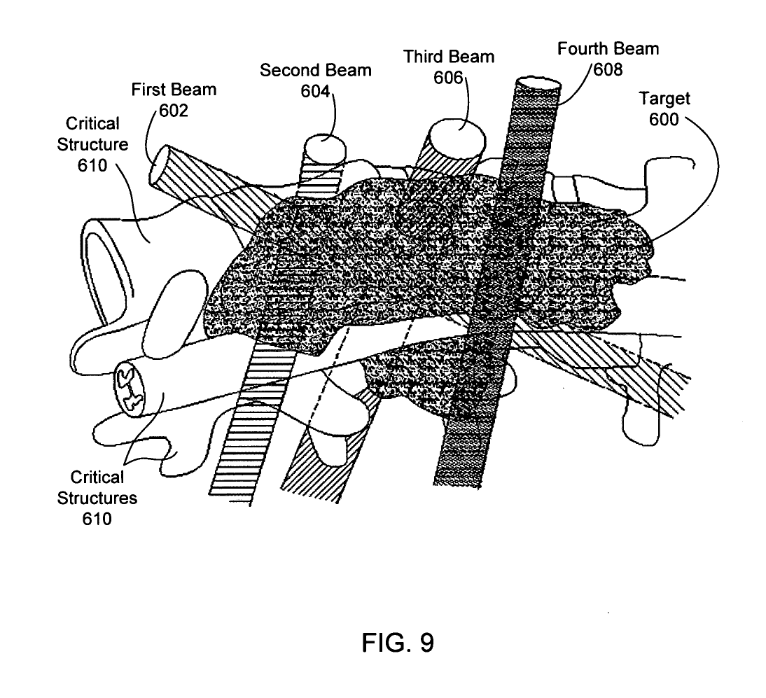

FIG. 9 illustrates a three-dimensional perspective view of a radiation treatment process. In particular, FIG. 9 depicts several radiation beams directed at a target 600. In one embodiment, the target 600 may be representative of an internal organ, a region within a patient, a pathological anatomy such as a tumor or lesion, or another type of object or area of a patient. The target 600 also may be referred to herein as a target region, a target volume, and so forth, but each of these references is understood to refer generally to the target 600, unless indicated otherwise.

The illustrated radiation treatment process includes a first radiation beam 602, a second radiation beam 604, a third radiation beam 606, and a fourth radiation beam 608. Although four radiation beams are shown, other embodiments may include fewer or more radiation beams. For convenience, reference to one radiation beam is representative of all of the radiation beams, unless indicated otherwise. Additionally, the treatment sequence for application of the radiation beams may be independent of their respective ordinal designations.

In one embodiment, the four radiation beams are representative of beam delivery based on conformal planning, in which the radiation beams pass through or terminate at various points within the target region 600. In conformal planning, some radiation beams may or may not intersect or converge at a common point in three-dimensional space. In other words, the radiation beams may be non-isocentric in that they do not necessarily converge on a single point, or isocenter. However, the radiation beams may wholly or partially intersect at the target 600 with one or more other radiation beams.

In another embodiment, the intensity of each radiation beam may be determined by a beam weight that may be set by an operator or by treatment planning software. The individual beam weights may depend, at least in part, on the total prescribed radiation dose to be delivered to the target 600, as well as the cumulative radiation dose delivered by some or all of the radiation beams. For example, if a total prescribed dose of 3500 cGy is set for the target 600, the treatment planning software may automatically predetermine the beam weights for each radiation beam in order to balance conformality and homogeneity to achieve that prescribed dose.

In the depicted embodiment, the various radiation beams are directed at the target region 600 so that the radiation beams do not intersect with the critical structures 610. In another embodiment, the radiation beams may deliver radiation treatment to the target region 600 by sweeping across the target region 600, as described above. The beam sweeping radiation treatment may be effectuated or facilitated by the relative movement between the target region 600 and the beam paths of the individual radiation beams.

It should be noted that the methods and apparatus described herein are not limited to use only with medical diagnostic imaging and treatment. In alternative embodiments, the methods and apparatus herein may be used in applications outside of the medical technology field, such as industrial imaging and non-destructive testing of materials (e.g., motor blocks in the automotive industry, airframes in the aviation industry, welds in the construction industry and drill cores in the petroleum industry) and seismic surveying. In such applications, for example, “treatment” may refer generally to the application of a beam(s) and “target” or “target region” may refer to a non-anatomical object or area.

The digital processing device(s) described herein may include one or more general-purpose processing devices such as a microprocessor or central processing unit, a controller, or the like. Alternatively, the digital processing device may include one or more special-purpose processing devices such as a digital signal processor (DSP), an application specific integrated circuit (ASIC), a field programmable gate array (FPGA), or the like. In an alternative embodiment, for example, the digital processing device may be a network processor having multiple processors including a core unit and multiple microengines. Additionally, the digital processing device may include any combination of general-purpose processing device(s) and special-purpose processing device(s).

Embodiments of the present invention include various operations, which may be performed by hardware components, software, firmware, or a combination thereof. As used herein, the term “coupled to” may mean coupled directly or indirectly through one or more intervening components. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Certain embodiments may be implemented as a computer program product that may include instructions stored on a machine-readable medium. These instructions may be used to program a general-purpose or special-purpose processor to perform the described operations. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; electrical, optical, acoustical, or other form of propagated signal (e.g., carrier waves, infrared signals, digital signals, etc.); or another type of medium suitable for storing electronic instructions.

Additionally, some embodiments may be practiced in distributed computing environments where the machine-readable medium is stored on and/or executed by more than one computer system. In addition, the information transferred between computer systems may either be pulled or pushed across the communication medium connecting the computer systems.

Although the operations of the method(s) herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative sense rather than a restrictive sense.

What is claimed is:

1. An apparatus, comprising:
a cup having a cavity configured to accept a breast of a patient within the cup; and
a fiducial marker coupled to the cup, the fiducial marker having a spatial relationship with a target region of the breast.

2. The apparatus of claim 1, further comprising an alignment marker on the cup to facilitate alignment of the cup on the breast in relation to an alignment landmark on the breast.

3. The apparatus of claim 2, wherein the alignment marker comprises an aperture and the alignment landmark comprises a nib attached to the breast.

4. The apparatus of claim 3, wherein the nib comprises a multi-modality fiducial.

5. The apparatus of claim 2, wherein the alignment marker comprises an aperture and the alignment landmark comprises a tattoo on the breast.

6. The apparatus of claim 2, wherein the alignment marker comprises an aperture to accept a nipple of the breast as the alignment landmark.

7. The apparatus of claim 2, further comprising a strap coupled to the cup to strap the cup on the breast, wherein the strap comprises a compression portion to compress an adjacent breast of the patient against a chest wall of the patient.

8. The apparatus of claim 1, wherein the cup is shaped to hold the breast in an immobilized position.

9. The apparatus of claim 1, further comprising a vacuum coupled to the cup to influence the breast away from a chest wall of the patient.

10. The apparatus of claim 9, further comprising a bolus material applied to the cup, wherein the bolus material is approximately between five and fifteen millimeters thick.

11. The apparatus of claim 1, wherein the cup comprises a radiolucent material to allow a treatment radiation beam to pass through the cup.

12. The apparatus of claim 11, further comprising a radiation source operably coupled with a stereotactic frame, the radiation source to generate the treatment radiation beam.

13. The apparatus of claim 12, further comprising a treatment planning system coupled to the radiation source to communicate control signals to the radiation source according to a treatment plan.

14. A method, comprising:
positioning a cup on a breast of a patient, wherein a fiducial marker is coupled to the cup; and
determining a relationship between the fiducial marker and a target region of the breast.

15. The method of claim 14, further comprising imaging the fiducial marker to determine a location of the fiducial marker relative to an imaging source.

16. The method of claim 15, further comprising:
positioning a radiation source relative to the target region based on a location of the fiducial marker; and
irradiating the target region with a radiation beam from the radiation source.

17. The method of claim 14, further comprising aligning the cup in an aligned position on the breast.

18. The method of claim 17, further comprising repositioning the cup on the breast in the aligned position after removal of the cup from the breast.

19. The method of claim 17, further comprising gluing a nib on the breast to align with a marking aperture in the cup, wherein the nib and the marking aperture align to indicate the aligned position of the cup on the breast.

20. The method of claim 17, further comprising marking a tattoo on the breast through a marking aperture in the cup, wherein the tattoo and the marking aperture align to indicate the aligned position of the cup on the breast.

21. The method of claim 14, further comprising aligning a marking aperture of the cup with a nipple of the breast to indicate the aligned position of the cup on the breast.

22. The method of claim 14, further comprising placing the cup on the breast while the breast is in a hanging position and the patient is in a prone position.

23. The method of claim 14, wherein the cup holds the breast in an elevated position away from a chest wall of the patient while the patient is in a supine position.

24. The method of claim 14, further comprising compressing an adjacent breast of the patient against a chest wall of the patient.

25. The method of claim 14, further comprising applying a vacuum to the cup to influence the breast away from a chest wall of the patient.

26. An apparatus, comprising:
means for immobilizing a breast of a patient in a protracted position away from a chest wall of the patient;
means for positioning an external fiducial marker near the breast; and
means for correlating a location of the external fiducial marker and a target region of the breast.

27. The apparatus of claim 26, further comprising means for reproducibly immobilizing the breast in an alignment position.

28. The apparatus of claim 26, further comprising means for influencing the breast away from the chest wall of the patient.

29. The apparatus of claim 26, further comprising means for flattening an adjacent breast against the chest wall of the patient.

30. The apparatus of claim 26, further comprising means for maximizing a number of potential radiation angles for a radiation source to irradiate the target region of the breast.
